(19) United States
(12) Patent Application Publication
(10) Pub. No.: US 2025/0049322 A1
(43) Pub. Date: Feb. 13, 2025

(54) AN APPARATUS AND METHOD FOR CAPTURING BIOMETRIC DATA FROM A HUMAN OR OTHER ANIMAL

(71) Applicants: Nicholas Roy GOMPERTZ, Oakham, Rutland (GB); Earswitch Ltd., Manchester (GB)

(72) Inventor: Nicholas Roy Gompertz, Manchester (GB)

(73) Assignee: Earswitch Ltd., Manchester (GB)

(21) Appl. No.: 18/718,089

(22) PCT Filed: Dec. 12, 2022
(86) PCT No.: PCT/GB2022/053172

§ 371 Date: Jun. 10, 2024

(30) Foreign Application Priority Data
Dec. 12, 2021 (GB) 2117939.5
Jul. 11, 2022 (GB) 2210188.5

(51) Int. Cl. A61B 5/00; A61B 5/0205

(52) U.S. Cl. CPC A61B 5/0075; A61B 5/0205; A61B 5/6817

(57) ABSTRACT

An apparatus for capturing biometric data from a human or other animal. At least part of the apparatus includes an ear portion configured to be receivable in an ear of the human or other animal. The ear portion includes a molecular spectroscopy device including at least part of an emitter and associated detector configured to capture biometric data relating to the molecular constituents of: blood passing through the ear of the human or other animal; and/or ear tissue of the human or other animal.

### CROSS-REFERENCE TO RELATED APPLICATION

This is a U.S. National Stage Application of PCT International Patent Application Number PCT/GB2022/053172, filed Dec. 12, 2022, which claims benefit of United Kingdom Patent Application No.: 2117939.5, filed Dec. 12, 2021 and United Kingdom Patent Application No.: 2210188.5, filed Jul. 11, 2022, the content of such applications being incorporated by reference herein in its entirety.

### FIELD OF THE INVENTION

The present invention relates to a device and method for measuring a multitude of biometric, physiological, health, fitness or other characteristics from a human or animal subject, enabling these characteristics to be detected during an application of the device to, or within, the ear-canal, and transmitted to a processor for output including display, storage, processing and transmission.

### BACKGROUND OF THE INVENTION

There is an increasing reliance on objective medical, health and fitness data from measurement of multiple biological and/or physiological and/or physical variables from people and animals to inform health and wellbeing, and medical, and veterinary decisions, management, treatment and lifestyle options. Subsequently this data, measurements or characteristics will be referred to as “biometrics”.

Medical biometrics including temperature, blood pressure, respiratory rate and pulse, are established measures of medical conditions and health. Current devices to record these biometrics in health care situations, or people's home environment, have relied on individual devices such as thermometers to measure temperature, blood pressure sphygmomanometer to measure pulse and blood pressure, and pulse oximeter to measure oxygen levels (oxygen saturations) and pulse.

There is no single device that measures multiple useful biometrics from one site for intermittent monitoring. Blood pressure sphygmomanometers also rely on inflatable cuffs (often around the upper arm) and are time intensive, inconvenient and uncomfortable, increasing the chance of a falsely high reading through the associated stress (white coat syndrome).

Biometrics can also be difficult or impossible to obtain by a healthcare, veterinarian or other operator. This can because of movement e.g. of an agitated subject such as a younger child, older person with delirium or an animal, or situation e.g. on scene at site of trauma or on battlefield where the subject is not positioned to be able to use a blood pressure cuff.

Oxygen saturations and respiratory rate can be particularly difficult to obtain in children under 5, who will often dislike the oxygen sensor, and unable to stay still or tolerate it for the time for the device to register a reading.

Despite this difficulty, it is usually possible for a child to remain still enough, often within their parent's arms, to use an ear-thermometer that is applied to the ear canal and takes a temperature from their tympanic membrane (ear-drum).

Additional biometrics such as measurements of heart function, and cognitive function in people with locked-in syndrome, are not currently available in community health care/primary care, or for other rapid near-patient assessment. No single medical device provides multiple biometric monitoring and auscultation of heart, chest and other physiological sounds.

Oxygen saturations taken from the finger or other peripheral locations are falsely lowered by the cold, poor circulation (e.g., physiological shock, or conditions such as Raynaud's), movement and non-white skin colour.

Oxygen saturation measurements are widely accepted globally, within medical guidelines, as an essential biometric for detecting and assessing life threatening illness, such as sepsis, COVID-19 and asthma. However, these are often difficult to obtain, may not be obtainable, and may be unreliable, and are widely accepted to incorporate a racial assessment bias because they are taken from areas of pigmented skin (for example the finger).

Respiratory rate in patients is difficult to record objectively during clinical examination in adults; informing a patient that their breathing rate is being counted may cause them to self-consciously alter their breathing rate. Covertly counting a patient's respiratory rate may be socially inappropriate as this requires the assessor to stare at the subject's chest silently, without first informing them of the reason, and so this action can be misconstrued. There is no current widely implemented method for objectively recording respiratory rate other than in intensive care or operating theatre environments.

There have been developments of biometrics within earpiece devices (wearables) for health, and fitness and medical monitoring, but not within diagnostic devices to aid with rapid assessment of biometrics for example in a clinical scenario.

Current earphone devices generally detect biometrics through a mechanism involving direct contact with the external ear, or external ear canal, which may be affected by cold, skin colour and movement. For example, PPG (photoplethysmography) sensors to detect pulse and oxygen levels are affected by movement, and the external canal moves during jaw movement, for example during screaming of an ill young child, or movement of an animal in a veterinary clinic.

One in-ear sensor has been Claimed (published as GB2585364) that detects central oxygen levels and biometrics from earphone sensors from the eardrum and surrounding structures, and provides for longitudinal monitoring in non-clinic scenarios in a wearable earphone device worn by the user. However, this sensor is limited if wax or other material obscures the ear-drum or surrounding structures from line of sight of the sensor.

Current devices are therefore limited in their ability to provide the wide range of accurate biometrics required in health care and veterinary care.

With respect to measuring oxygen saturation, current methods may be summarised as: 1) standard oximetry; and 2) video or remote PPG. Standard oximetry utilises one photodiode and two light emitting diodes (LEDs), one infrared and one red. The LEDs alternately illuminated, and the photodiode is responsive to wavelengths from both LEDs. A ‘ratio of ratios’ is generated from the output according to the wavelength of each LED. This requires direct skin contact and is, therefore, affected by movement, skin colour, etc. Video or remote PPG utilises a standard camera to look for differences in colour of a facial image with flushing of blood. It is understood that this uses a ratio of red against green (or other colour of light) to provide a pulse waveform and oxygen saturation. As this does not use light of a defined wavelength, as one would with LEDs, this method is affected and dependent upon external light. It is also affected by facial, head, camera movement(s), etc.

With respect to Raman spectroscopy, directing an emitter to and, thereby, detecting reflected signals from an ear-drum and/or malleus produces excessive signal fluorescence from the bony structure of the malleus abutting the ear-drum, which saturates a detector, preventing detection of the desired Raman signal.

### SUMMARY OF THE INVENTION

According to a first aspect, the present invention provides an apparatus for capturing biometric data from a human or other animal, at least part of the apparatus comprises an ear portion configured to be receivable in an ear of said human or other animal, wherein the ear portion comprises molecular spectroscopy means comprising at least part of an emitter and associated detector configured to capture biometric data relating to the molecular constituents of:
blood passing through the ear of said human or other animal; and/or
ear tissue of said human or other animal.

Preferably, the spectroscopy means is Raman spectroscopy means, an emitter of which is configured to (only) illuminate the medial ⅓^{rd} or ⅔^{rds} of the ear-canal.

Preferably, the ear portion further comprises an imager for assisting in targeting only the medial ⅔^{rds} of the ear-canal 7′ for Raman spectroscopy.

Preferably, the detector is configured to capture biometric data from the:
ear drum;
inner (medial) section of the ear canal;
the outer (lateral) section of the ear canal;
middle ear; and/or
any combinations thereof.

Preferably, the apparatus additionally comprises one or more sensor means configured to capture biometric data relating to one or more of the group comprising:
sound, preferably, breathing sounds and/or cardiovascular sounds, for example, heart sounds, murmur, bruits, blood flow and/or voice;
motion, preferably, gait, cardiac output impulse (as measured for ballistocardiography), tremor or spasms;
temperature, preferably, tympanic temperature;
blood pressure, preferably, pulse transit time and/or pulse arrival time;
respiratory rate;
respiratory rhythm and/or character;
pulse;
oxygen saturation;
appearance of ear structures, eardrum and/or ear contents, preferably, one or more images or videos thereof;
heart function, preferably, pre ejection period and/or electrocardiograph;
ear-drum response to sound; and/or
intentional and/or voluntary movement of middle ear muscles.

Preferably, the apparatus comprises one or more sensor means configured to capture biometric data from an external ear, pinna, neck, chest, hand, or digit. Preferably, biometric data is captured through skin contact or imaging, but this is not essential. Preferably, non-ear sensors comprise a microphone, ECG terminals, a touch sensor, and/or a motion sensor.

Preferably, the ear portion of the apparatus additionally comprises one or more sensor means configured to capture biometric data relating to one or more of the group comprising:
sound, preferably, breathing sounds and/or cardiovascular sounds, for example, heart sounds, murmur, bruits, blood flow and/or voice;
motion, preferably, gait, cardiac output impulse (as measured for ballistocardiography), tremor or spasms;
temperature, preferably, tympanic temperature;
blood pressure, preferably, pulse transit time and/or pulse arrival time;
respiratory rate;

respiratory rhythm and/or character;
pulse;
oxygen saturation;
appearance of ear structures, eardrum and/or ear contents, preferably, one or more images or videos thereof;
heart function, preferably, pre ejection period and/or electrocardiograph;
ear-drum response to sound; and/or
intentional and/or voluntary movement of middle ear muscles.

Preferably, the one or more sensor means comprise an emitter and associated detector.

Preferably, the apparatus further comprises: processor means, for analysing the biometric data, and/or display means, for displaying one or more biometrics of said human or other animal.

Preferably, the apparatus is an earphone, earbud, or ear-mounted apparatus, which may comprise an external ear portion.

Preferably, the external ear portion is configured to be mounted in or to the external ear or pinna.

Preferably, the apparatus comprises a hand-held portion in addition to the ear portion, the portions being operatively connected for communication therebetween.

Preferably, the apparatus comprises: an integrated hand-held portion capable of communicating directly with the ear portion; or a separate hand-held portion capable of communicating wirelessly or by wired communication with the ear portion.

Preferably, the hand-held portion comprises a display means. Alternatively, or in addition, display means may be provided by an associated computer, smart phone and/or digital display, or the like.

Preferably, one or both of the hand-held portion and/or the ear portion comprise(s) processor means for analysing the biometric data.

Preferably, the at least part of the emitter and associated detector are configured to detect a reflected signal from the medial ⅔^{rds} of the ear-canal.

Preferably, the at least part of the emitter and associated detector are configured to only detect a reflected signal from the medial ⅔^{rds} of the ear-canal.

Preferably, the at least part of the emitter and associated detector are configured to (only) detect a reflected signal from the medial ⅓^{rd} of the ear-canal.

Preferably, the emitter is configured so as to not directly illuminate the ear-drum nor malleus. Most preferably, the emitter is configured to illuminate the medial ⅔^{rds} of the ear-canal, or medial ⅓^{rd} of the ear-canal, without illuminating the ear-drum and/or malleus.

Most preferably, emitter is configured to illuminate: an ear-canal region of increased blood flow; and/or non-pigmented skin of the ear-canal.

Preferably, the apparatus comprises means for securely fastening the ear portion within the ear-canal of a user. Most preferably, the means for securely fastening comprises an anchor, for securely fastening the apparatus to an external ear of said user.

Preferably, the ear portion comprises adjustment means for controlling and/or locking in situ a position of the ear portion, at least part of the emitter and/or associated detector.

According to a second aspect, the invention provides a method for capturing biometric data from a human or other animal, the method comprising:
locating at least part of an apparatus in an ear of the human or other animal, so as to locate at least part of an emitter and detector of molecular spectroscopy means in the ear;
utilising the molecular spectroscopy means to capture biometric data relating to the molecular constituents of:
   blood passing through the ear of the human or other animal; and/or
   ear tissue of said human or other animal.

Preferably, the method comprising utilising Raman spectroscopy to capture biometric data and illuminating the medial ⅔^{rds} of the ear-canal.

Preferably, the method comprising analysing the biometric data within the ear portion, an earphone, earbud, or ear-mounted apparatus.

Preferably, the method comprising analysing the biometric data within a hand-held portion of the apparatus.

Preferably, displaying one or more biometrics of the human or other animal.

Preferably, the method comprising communicating biometric data to an external processor for analysing and displaying one or more biometrics of the human or other animal.

Preferably, the emitter of the spectroscopy means emits towards the:
ear drum;
inner (medial) section of the ear canal;
the outer (lateral) section of the ear canal;
middle ear; and/or
any combinations thereof.

Preferably, the method comprising detecting biometric data relating to one or more of the group comprising:
sound, preferably, breathing sounds and/or cardiovascular sounds, for example, heart sounds, murmur, bruits, blood flow and/or voice;
motion, preferably, gait, cardiac output impulse (as measured for ballistocardiography), tremor or spasms;
temperature, preferably, tympanic temperature;
blood pressure, preferably, pulse transit time and/or pulse arrival time;
respiratory rate;
respiratory rhythm and/or character;
pulse;
oxygen saturation;
appearance of ear structures, eardrum and/or ear contents, preferably, one or more images or videos thereof;
heart function, preferably, pre ejection period and/or electrocardiograph;
ear-drum response to sound; and/or
intentional and/or voluntary movement of middle ear muscles.

Preferably, the method comprising detecting biometric data from an outer ear, pinna, neck, chest, hand, or digit.

Most preferably, (only) capturing biometric data from the medial ⅔^{rds} of the ear-canal, or medial ⅓^{rd} of the ear-canal, preferably, without illuminating the ear-drum and/or malleus.

Preferably, the method comprising any one or more features of the first aspect.

According to a third aspect, the invention provides an apparatus for capturing biometric data from a human or other animal, at least part of the apparatus comprises an ear portion configured to be receivable in an ear of said human or other animal, wherein the ear portion comprises sensor means comprising at least part of an emitter and associated detector of a photoplethysmography sensor means configured to be directed to non-pigmented ear tissue so as to capture biometric data from said non-pigmented ear tissue.

Preferably, the photoplethysmography sensor means comprises a non-contact photoplethysmography sensor means. Most preferably, the photoplethysmography sensor means is, in use, directed to non-pigmented ear tissue without touching it.

Preferably, the apparatus comprises one or more sensor means configured to capture biometric data relating to:
sound, preferably, breathing sounds and/or cardiovascular sounds, for example, heart sounds, murmur, bruits, blood flow and/or voice;
motion, preferably, gait, cardiac output impulse (as measured for ballistocardiography), tremor or spasms;
temperature, preferably, tympanic temperature;
blood pressure, preferably, pulse transit time and/or pulse arrival time;
respiratory rate;
respiratory rhythm and/or character;
pulse;
oxygen saturation;
appearance of ear structures, eardrum and/or ear contents, preferably, one or more images or videos thereof;
heart function, preferably, pre ejection period and/or electrocardiograph;
ear-drum response to sound; and/or
intentional and/or voluntary movement of middle ear muscles.

Preferably, the sensor means is configured to capture biometric data from the:
ear drum;
inner (medial) section of the ear canal;
the outer (lateral) section of the ear canal;
middle ear; and/or
any combinations thereof.

Preferably, the apparatus is configured to detect biometric data from an outer ear, pinna, neck, chest, hand, or digit. Preferably, non-ear sensors comprise a microphone, ECG terminals, a touch sensor, and/or a motion sensor.

Preferably, the apparatus comprises a plurality of sensor means, in which at least two sensor means are configured to detect the same biometric data from different parts of the ear.

Preferably, the two different parts of the ear are selected from the ear drum, inner (medial) section of ear canal, outer (lateral) section of ear canal, and/or middle ear.

Preferably, the apparatus comprises means for securely fastening the ear portion within the ear-canal of a user. Most preferably, the means for securely fastening comprises an anchor, for securely fastening the apparatus to an external ear of said user.

Preferably, the apparatus additionally comprises molecular spectroscopy means, at least part of which is located in the ear portion for capturing biometric data relating to the molecular constituents of:
blood passing through the ear of said human or other animal; and/or
ear tissue of said human or other animal.

Preferably, the photoplethysmography sensor means is configured to emit and detect multi-wavelength and/or white light.

Preferably, the detector comprises an RGB camera or RGB-IR camera.

Preferably, the photoplethysmography sensor means is configured to emit constantly.

Oxygen saturation measurements may be provided by emitting multiple wavelength light (e.g. white light) and detected using a multi-colour detector (e.g. an RGB (red, green, blue) camera). A comparison of reflected red-light—associated with degree of saturation of haemoglobin with oxygen—against another wavelength of light using a ‘ratio of ratios’ analysis provides a percentage of oxygen saturation. Preferably, the detector (e.g. camera) and light source (e.g. white light LED) are provided within the ear-canal, at a distance (stand-off) from the ear-drum/ear-margin.

Advantageously, a standard camera may be used, which is relatively inexpensive and/or small, and readily available. Only one LED (white) is required. Owing to stand-off, the apparatus is less affected by movement. The LED is constantly illuminated during detection—as compared to flickering LEDs on/off may cause variable signals as LEDs light-up and warm-up. In an alternative, constant illumination is proposed for a standard oximetry emitter, having one red and one infrared LED, in combination with an RGB-IR camera—being a red, green, blue camera which is IR sensitive, or other sensor differentially sensitive to multiple wavelengths of light.

Preferably, the apparatus is located in a tube which may be insertable in an ear of an individual. Alternatively, the tube may be used simply to provide detection without external light by a person placing a finger, palm or other over an end of the tube to, thereby, prevent external light getting to the camera and LED placed at some distance from the end of the tube. Advantageously, the apparatus can provide detection in ambient darkness, as it does not rely upon external light.

Preferably, the apparatus comprises any one or more features of the first aspect.

According to a fourth aspect, the invention provides a method for capturing biometric data from a human or other animal, the method comprising:
locating at least part of an apparatus in an ear of the human or other animal, so as to locate at least part of an emitter and detector of photoplethysmography sensor means in the ear;
directing the emitter and/or detector to non-pigmented ear tissue; and
detecting and/or recording biometric data from said non-pigmented ear tissue.

Preferably, the method comprises detecting and/or recording biometric data relating to:
sound, preferably, breathing sounds and/or cardiovascular sounds, for example, heart sounds, murmur, bruits, blood flow and/or voice;
motion, preferably, gait, cardiac output impulse (as measured for ballistocardiography), tremor or spasms;
temperature, preferably, tympanic temperature;
blood pressure, preferably, pulse transit time and/or pulse arrival time;
respiratory rate;
respiratory rhythm and/or character;
pulse;
oxygen saturation;
appearance of ear structures, eardrum and/or ear contents, preferably, one or more images or videos thereof;
heart function, preferably, pre ejection period and/or electrocardiograph;
ear-drum response to sound; and/or
intentional and/or voluntary movement of middle ear muscles.

Most preferably, the photoplethysmography sensor means is located in a spaced and non-touching relationship with respect to the non-pigmented ear tissue.

Preferably, the photoplethysmography sensor means emits and detects multi-wavelength and/or white light.

Preferably, the method comprises utilising an RGB camera or RGB-IR camera.

Preferably, the photoplethysmography sensor means emits constantly during detection.

Preferably, the method is conducted on an apparatus according to the third aspect.

Preferably the method comprises any one or more features of the second aspect.

The present invention is a device and method for measuring a multitude of biometric, physiological, health, fitness or other characteristics from a human or animal subject, enabling these characteristics to be detected during an application of the device to, or within, the ear-canal, and transmitted to a processor for output, including display, storage, processing and transmission of biometric data.

No current single device exists that provides a range of reliable commonly obtained biometrics, with the additional potential benefit of providing near patient recording of other biometrics, such as cardiac contractility and in-ear photography, and electrocardiogram (ECG) monitoring.

The invention is a device and method to be applied to, or near the subject's ear-canal, with a multitude of sensors from which data is derived, which are able to provide information on biometrics which may include: blood pressure, pulse wave form, pulse rate and rhythm, heart rate variability, ECG, oxygen saturations, pulse arrival time (PAT), pulse transit time (PTT) (as surrogate for cuffless blood pressure measurements), pre-ejection period (PEP) (as a measurement of cardiac contractility), respiratory rate, temperature, photograph and/or video of ear canal and other ear structures, and response of ear-drum to emitted sound, and intentional and involuntary movement.

Sensors may include a plurality of sensors directed at the ear-drum, and/or surrounding structures, any position within the ear canal, including the inner (medial) ear-canal wall or outer (lateral) ear-canal wall, within, or on, the pinna and on the neck, hand, finger or any other body location

Sensors may include a plurality of sensors including; MEMS (micro electro mechanical systems) or other microphones, ultrasound transducers, optical sensors (including PPG, LiDAR, self mixing interferometer, time of flight, laser, proximity, of any visual or non-visual wavelength, including infrared), thermocouple or other temperature sensor, movement sensors, including inertial measurement unit (IMU), and/or MEMS gyroscope sensor and/or accelerometer or other sensor with ability to detect movement, including those related to ballistocardiography measurements (BCG), electrodes capable of detecting electrocardiogram (ECG), electroencephalogram (EEG), electrooculogram (EOG) signals, camera and/or any movement detector and/or proximity and/or capacitance sensor (including Saw Wave resonator), or radio frequency &/or RADAR transducers.

The device may include emitters, including emitters of light, including LED and laser of any visible or non-visible light wavelength, sound emitter, ultrasound emitter, or radiofrequency emitter, or other emitter. The device may include method of introducing fluid or air to the ear-canal, or of removing substances such as wax, fluid, pus or other material, which may include a port or aperture for transmission of material, fluid, suction, air or apparatus.

The device may consist of a handheld device of any shape, applied to, or near, the ear canal by an operator or the subject themselves, or of a device that is applied to, or near, the ear-canal and connected to another device by any wired or flexible connection, or be applied to, or near, the ear-canal and communicate wirelessly or by any other method to another device, either synchronously or asynchronously.

Further embodiments include two devices; one applied to, or near, the ear canal of each ear, and connected to each other and/or another device, by wired, wireless or any other connection, which may be synchronous or asynchronous.

Embodiments include the non-ear related aspect of the device attached to a lanyard, or other material, to be worn around the neck, similar to a pendant, enabling handsfree measurements, including movement detection from sensor in the device suitable to detect movement appropriate for BCG wave analysis.

Other embodiments include the sensors on a handheld, and/or non-ear related aspect of the device, with multiple surface electrodes, which may be positioned along an aspect of the device, suitable to be applied to the chest wall, at a site positioned to detect electrical activity from the heart (for example several ECG “chest leads”).

The sensors may detect biometrics directly, or from a distance, for example light transmitted to any optical sensor via guides or channels, such as fibre-optics. Such sensors or optical guides may be directed to any area within or near the eardrum or ear canal, including inner or outer sections of the ear canal.

Outputs from the sensors may be transmitted to processor or processors of embodiments.

Embodiments include the device containing, or being connected remotely, or directly to a processor. The processor may be within an earpiece structure, and/or within a hand-held structure, or other structure, which may be physically connected to the earpiece or remotely. The device may be attached to the processor by wired or wireless connection. The processor or attached processor may apply algorithm to the data which may be generated from machine learning, and transmit and/or display the output data generated by the algorithm.

Embodiments include outputs of the processor dependent on algorithms of the processor which may be predefined, user defined, or altered and/or developed using pre-existing or developing machine learning processes. The output of the processor, which may be affected by algorithms of embodiments, may be communicated through wire, wirelessly and/or remotely, synchronously, asynchronously and/or stored on electronic media. In embodiments the communicated output data may be presented to an individual, processor or graphical, or other, user interface or display, as data which may be interpreted and/or presented representing biometric data and/or other data.

The device may include a display that provides a representation of the biometrics or/and connect to a device that displays and/or records and/or transmits the data.

The device may be powered by a battery, wired connection or other power source, including self-contained generator within the device. An embodiment may include a power source of the device being a generator within the handle operated by a user pumping an expandable handle of the device.

Embodiments may, for example, be a hand-held device, similar in size and shape to a tympanic thermometer with a structure suitable to apply into the ear-canal. This structure may include one or many sensors. These sensors may include any or all of LiDAR, self mixing interferometer, camera and/or thermometer sensors and/or PPG sensors (including for pulse oximetry), ultrasound transducers, MEMS microphone, MEMS speaker, with sensors directed at the ear-drum, and/or ear canal and/or surrounding structures. PPG sensors may also be directed at the external ear canal.

EEG, EOG and ECG electrodes may be positioned to contact the ear canal, and/or pinna, and on the handle, or other aspect of a device, for the subject to touch and/or hold, and/or an extension or aspect of the device incorporating electrodes and/or sensors to contact the subject's neck, or contact or be attached to any point on the body, and connected to the device by a wire or wirelessly. Embodiments include those containing sensors within the ear applied device, or wired or wirelessly connected device, that are IMU and/or other sensors capable of detecting movements relevant to ballistocardiography measurement.

In embodiments the device may be configured to record video and/or photography of the ear-drum (including oil or fluid enhanced imaging), tympanic temperature, oxygen saturations, respiratory rate, pulse rate and rhythm, pulse waveform, heart sound, breathing sounds, carotid bruits, PEP, PAT, PTT, ECG, EOG, EEG, ear-drum mobility (in response to emitted sound, and tremor, and related to eye movement and voluntary control, in relationship to pressuring the canal), and allow removal, for example by micro suction of wax, pus and other material.

A sensor in the base/foot, or other aspect, of the device may also be positioned which may be one or more of any of; microphone, PPG and/or movement detector, that may detect vascular or other movements, and/or sounds, including carotid pulsation, jugular venous pressure wave and breathing and/or laryngeal and/or voice sounds and/or movements.

Embodiments include output of processors of devices providing an immediate display of multiple biometrics, including a cuffless blood pressure measurement, and also an assessment of cardiac contractility by a single application of the diagnostic device to the subject.

Pulse arrival time (PAT) and Pulse transit time (PTT) are cardiovascular measurements calculated by comparison of timing of various measures of cardiac contraction, with arrival of pulse pressure at more peripheral body sites. PAT and PTT are both cuffless surrogate measures for blood pressure. Pre-ejection period (PEP), is a measure calculated by comparison of ECG data and cardiac ejection which may be calculated from BCG waveform. PEP is a measure of cardiac contractility.

In embodiments the algorithm of the processor will generate measurements of any of PAT, PTT, PEP and other cardiovascular measurements. This may include incorporation of data from ECG electrodes, and/or auscultated heart sounds, carotid pulse, and/or PPG waveform at the ear-drum, ear-canal and/or neck, or any sensor data input.

The embodiments presented here and other embodiments provide multiple opportunities to detect, record, measure and analyse multiple relationships between biological and cardiological variables. These include but are not limited to relationships between ECG characteristics (including P, Q, R, S, T, U waves, and/or onset of electrical cardiac activity), cardiac “heart sounds” (1st, 2nd, 3rd, split, murmurs and any other cardiac sounds), BCG waveforms (any BCG wave), pulse waveforms from ear-canal, ear-drum, neck, chest or other site (to include but not limited to carotid waveform). These may give insight into biological relationship between heart contraction, cardiac valve sounds and/or function (for example mitral, tricuspid, aortic and/or pulmonary valve opening), and relative pressure, for example, in atria, ventricles, aortic root, pulmonary artery and aortic valve pressure gradient.

Inclusion of multiple different sites and types of sensors detecting cardiac characteristics allows algorithm of the processor to enhance signal to noise ratio for biometrics, for example those which represent a surrogate measure for cuff-less blood pressure.

Comparison of PPG traces from several sites, which may include the ear-drum, ear canal or other site, including finger and/or neck, may provide additional health related information. This may provide a comparison of pulse waveform characteristics and timings between pulse data derived from internal carotid circulation (from the ear-drum vascular anastomosis), and from the external carotid (from the ear-canal), or other vascular circulation. This analysis of differential flow between internal and external carotid blood territories may be affected by stenosis of the internal carotid, or distal blockage of large blood vessels such as the middle cerebral artery in thrombotic stroke, and present a novel new medical measurement.

The embodiment comprising a device positioned within or near the ear-canal, either flexibly connected or separate from another device, allows detection of the cranial direction (vertical) pulsatile movement of the body due to ejection of the blood from the heart, without the movement and/or signal being dampened by a user holding the device. This provides a more robust and reliable BCG signal from which to generate PAT, PTT and PEP measurements.

Other embodiments of the device incorporating microphones provide additional functions, which may include recording, amplification, transmission and interpretation of sounds including detected from the ear canal, at the neck, chest or other site. This may give information related to carotid blood flow, including carotid bruits, breath sounds, laryngeal sounds, heart sounds or other sounds. The sensor output related to these sounds may be analysed by the algorithm of the processor in conjunction with other sensor data, and provide output related to the timings of the cardiac cycle, for example PEP, PAT, PTT and blood pressure.

These microphones may be positioned in a handheld device incorporating an ear related module, or a separate device that may be in connection, by wired or wireless method, with the ear-related device.

An embodiment may incorporate a microphone, or other sound or movement sensor, including positioned at the base, or other aspect of a handheld device, and act as a stethoscope within the device, transmitting the sound to the user's ears, and/or recording, and/or transmission and/or interpretation, with any algorithm including machine learning based. This may incorporate information on cardiac cycle timing of cardiac sounds related to other sensor output, for example electrodes providing data related to ECG signals.

Embodiments including a camera within the device facing the eardrum, will enable photographs and/or videos of the ear-drum/ear-canal or its contents, or any other skin, or other body feature, to be recorded, transmitted, viewed and/or interpreted. This will allow detailed review of features after the application of the device, which is advantageous over currently widely used optical otoscopes which only allow live views, and may be transient in a moving younger child or animal. It also has advantage in providing this view/information contemporaneously to gathering other biometrics. Cameras in other embodiments with lens, and/or optical qualities for magnification may provide ear-drum microscopy providing images and video of micro-vasculature of the ear-drum.

The eardrum vibrates in response to sound received by the ear, and this is known to be detected by laser detection devices such as laser Doppler vibrometry. Some conditions affect the ability of the eardrum to vibrate sufficiently, including perforations and otosclerosis. An embodiment whereby a sound (“test sound”) is emitted from the device within or close to the ear-canal, and a sensor (for example a LiDAR sensor) detects vibration, and/or movement, and/or lack of movement, of the eardrum in response to this, will provide data that may be used to provide information on whether the ear-drum movement is normal. This may be used to configure amplitude and frequency of emitter settings on hearing aids to aid improved hearing for people with these conditions.

Some people are able to voluntarily move a muscle in their middle ear, and detection by this device of this voluntary movement will indicate the ability of the subject to understand commands for example, those with locked-in syndrome caused by brain stem stroke.

Some medical conditions cause involuntary movements including tremors and spasm. One embodiment of this device includes the sensor of the device detecting movement, tremors or spasms of the ear-drum muscle, related to movement or spasm of the tensor tympani itself, or reflecting movement or spasm of the eye muscles or any other muscle movement.

This invention provides output of biometric measurements that are more robust than current device output, by incorporation of output data from multiple sensors that may be affected differently between signal and noise (for example IMUs and PPGs and other movement sensors). This allows algorithms of the processor to improve signal to noise ratio by combining data from more than one sensor type or site, for example by reducing movement artefact.

Benefits over existing devices for intermittent monitoring, also include collection of oxygen saturation data from structures that are not pigmented in any racial groups, for example from the eardrum, adjacent structures and/or inner (medial) section of the ear canal wall. This provides accuracy for people of racial groups with dark skin colour, overcoming a limitation of current devices. Current embodiments of the invention, including sensors directed at the medial ear canal wall, provide advantage over ear-drum sensors when the direct line of sight from the sensor to the ear-drum is obstructed. This current invention is more robust to ear wax or debris or other material than other ear-drum directed sensors. Central (core) oxygen saturation measurements are not affected by cold peripheries, or poor peripheral blood circulation or movement, and so are more accurate than existing peripheral oxygen saturations monitors.

The current invention provides a multi-modal biometric measurement device which provides extensive increase functionality and additional new biometrics within devices useful in the clinical and home environment that have not been previously claimed.

This device will be the first device to give reliable measurement of oxygen saturations in all ethnic groups, provides the first easily applied cuffless blood pressure measure, provide easier and quicker assessment of biometrics for patients in all groups, and also in those hard to assess (e.g., young children, agitated patients, and veterinary patients), and novel biometrics (response of ear drum, movement, comparison of PPGs). Machine learning data analysis will facilitate new insights to relationships between different biometrics patterns and health.

Earphone devices do not provide this breadth of biometrics and rely on user compliance, whereas the ear-thermometer platform is well known to be able to detect temperature even in agitated younger children, enabling oxygen saturations and respiratory rate to be obtained reliably.

Advantageously, the invention provides PPG detection in combination with ballistocardiography in the ear (an accelerometer to detect impulse effects on the head of blood ejected from the aorta) to provide timings for pulse transit time measurement—this being a cuffless surrogate for blood pressure detection.

Advantageously, the invention provides ECG detection with BCG, heart sounds and carotid sounds (heard via a microphone in the ear or externally applied to the chest or carotid artery) to provide other cardiac insights, including pre-ejection period which is a measure of cardiac contractility.

Advantageously, the invention provides ‘heat maps’, the ability to compare: distribution of blood flow within the ear, for example by comparing a ‘perfusion index’ pixel-by-pixel, and/or comparing pulse wave forms within a frame or against other PPG signals; and/or distribution of oxygenation within the ear.

Advantageously, the invention provides spectroscopy, as an indicator of wax (yellow or similar colour)—for example, to provide automated or self-diagnosis for a hearing aid equipped with the sensor or by a hand-held monitoring device.

Advantageously, the invention provides intermittent Raman/biomolecular monitoring during normal daily living when mounted in ear-phones. Further advantageously, when stable and securely mounted, the detection is unaffected by chewing/talking.

Advantageously, the invention provides the ability to correlate data and provide insight for biomolecular monitoring with physiological monitoring, to aid disease monitoring and obtain disease insights.

Advantageously, the invention can compare ear-drum/ear-drum margin to outer ear-canal PPG.

The term ‘illuminate’ is used in connection with any form of electromagnetic radiation, and not just visible light.

### BRIEF DESCRIPTION OF THE DRAWING FIGURES

The invention will now be disclosed, by way of example only, with reference to the following drawings, in which:

FIG. 1 is a pictorial representation of a cross section of the right ear canal and partial view of middle ear with one embodiment of the invention of a handheld device directed towards the ear-canal;

FIG. 2 is a pictorial representation of an embodiment of the invention as device with an in-ear sensor module with a flexible attachment to a handheld module, and remote display;

FIG. 3 is a pictorial representation of the graphical output of measurements derived from the invention;

FIG. 4 is a diagram showing indicative forms of data that can be detected from and transmitted from the sensors of the embodiments of the device of the invention; and

FIG. 5 is a schematic diagram of an ear-based apparatus.

### DETAILED DESCRIPTION OF THE INVENTION

The present invention is a device and method for measuring a multitude of biometric, physiological, health, fitness or other characteristics from a human or animal subject, enabling these characteristics to be detected during an application of the device to, or within, the ear-canal, and transmitted to a processor for output including display, storage, processing and transmission.

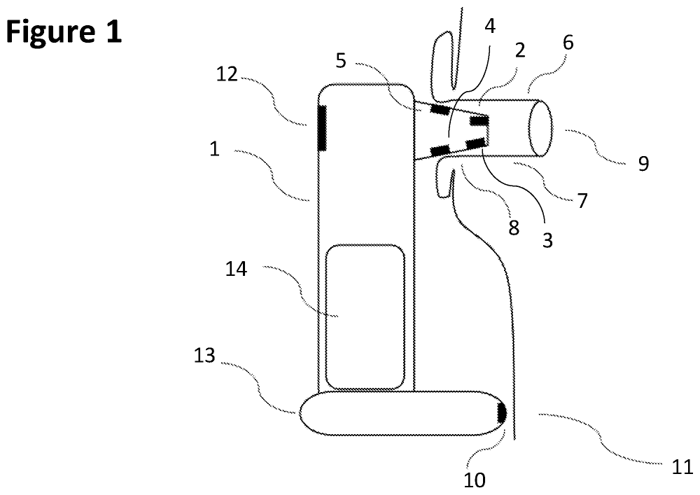

FIG. 1 illustrates one embodiment of a handheld device 1, incorporating several sensors 2,3,4,5, as a cross sectional view of the right ear canal 6. The sensors being located in the ear canal 6 and directed towards the ear drum (tympanic membrane) 9, the medial (inner) section of the ear canal 7, and/or the lateral (outer) section of the ear canal 8. In a preferred version, where one of the sensors 2, 3, 4, 5 is a Raman spectroscopy sensor, an emitter of which is configured to illuminate only the medial ⅔^{rds} of the ear-canal.

The sensors 2,3,4,5 may have a configuration including imagers 2, which may be a video camera, or an infrared video camera, with or without light source, or have a laser emitting and receiving combination, and/or PPG sensors, and/or function as Raman spectroscopy transducers, or any combination of sensors, including but not limited to temperature sensors. The device may have one or more electrodes, 5, in contact with the ear canal 6 or external ear (pinna), and one electrode, 10, on the base of the handle of the device 13 able to be in contact with skin of the neck 11, or on outer aspect of the device 12, suitable to be touched by the subject's finger, to make a contact for detecting an electrical signal, for example an ECG signal. In one embodiment a sensor 10, in contact with the neck, may be a microphone, and/or PPG sensor, or other movement or sound detection sensor, that detects pulsation or movement, including pulsation of the carotid artery, or jugular vein. The embodiment in FIG. 1 demonstrates a visual graphical display 14 on the handle of the device 1 to exhibit representations of the measurements detected by the sensors (2,3,4,5, 10, 12).

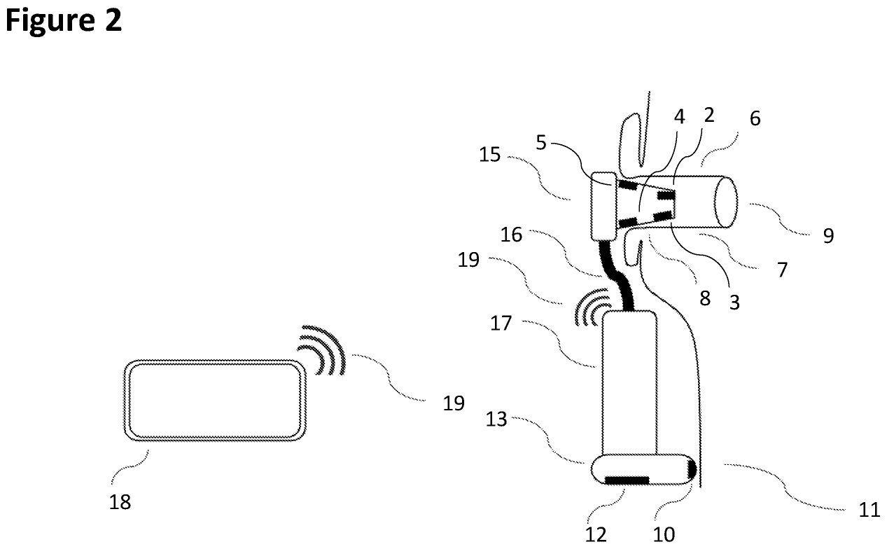

FIG. 2 illustrates a further embodiment of a handheld device 17, incorporating an earpiece component 15, connected by flexible component 16, for example a wire or other more robust component, to a handheld device 17, or other component. The earpiece component 15 incorporates several sensors 2,3,4,5, as described above for FIG. 1. The sensors being located in the ear canal 6 and directed towards the ear drum (tympanic membrane) 9, the medial (inner) section of the ear canal 7, and the lateral (outer) section of the ear canal 8. In a preferred version, where one of the sensors 2, 3, 4, 5 is a Raman spectroscopy sensor, an emitter of which is configured to illuminate only the medial ⅔^{rds} of the ear-canal. The sensors 2,3,4,5 may have a configuration including imagers 2, which may be a video camera or an infrared video camera, with or without light source, or have a laser emitting and receiving combination, and/or PPG sensors, and/or function as Raman spectroscopy transducers, or any combination of sensors, including but not limited to temperature sensors. The device may have one or more electrodes 5, in contact with the ear canal 6 or pinna, and one electrode 10, on the base of the handle of the device 17 to be in contact with skin of the neck 11, or on an outer aspect of the device 12, suitable to be touched by the subject's finger, to make a contact for detecting an electrical signal, for example an ECG signal. This embodiment demonstrates a remote visual graphical display 18, for example a computer display, to exhibit representations of the measurements detected by the sensors (2,3,4,5,10,12), showing the data being communicated wirelessly 19 to the processor of the display. Variations of this embodiment include a handheld device 17, or other component, attached to a lanyard, and worn around the neck of the subject in a similar manner to wearing a pendant, and/or incorporating multiple electrode sensors suitable to contact multiple sites synchronously across the chest. In a further variant, the other component is a desk-mounted device.

The flexible component may include fibreoptics, such that one or more sensors are located in the handheld device, other worn device, or the desk-mounted device, and the fibre-optics provide an output to an associated emitter and/or an input to an associated detector of the sensor, such that the one or more sensors do not themselves require location in the earpiece component.

This embodiment includes versions incorporating two of the earpiece components 15, one in each ear, connected via flexible component 16, to each other, and/or a handheld device 17, or other component.

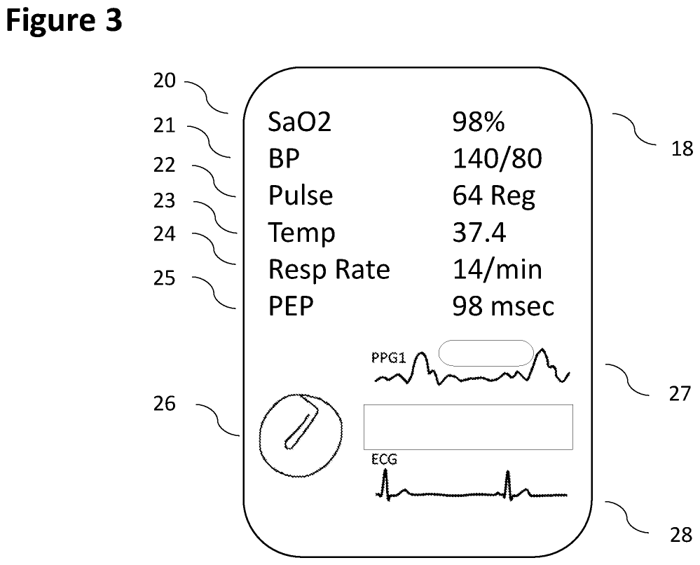

FIG. 3 illustrates the display 18 of embodiments of the invention demonstrating some of the biometrics that may be displayed, including central oxygen saturations (SaO2) 20, blood pressure (BP) 21, (derived from PAT and or PTT), pulse rate and pulse rhythm 22, temperature 23, respiratory rate 24, pre-ejection period (PEP) 25, image of the eardrum 26, pulse waveform 27 from PPG sensors, and ECG monitoring 28. Embodiments may include any configuration of display options including multiple or single biometrics displayed synchronously.

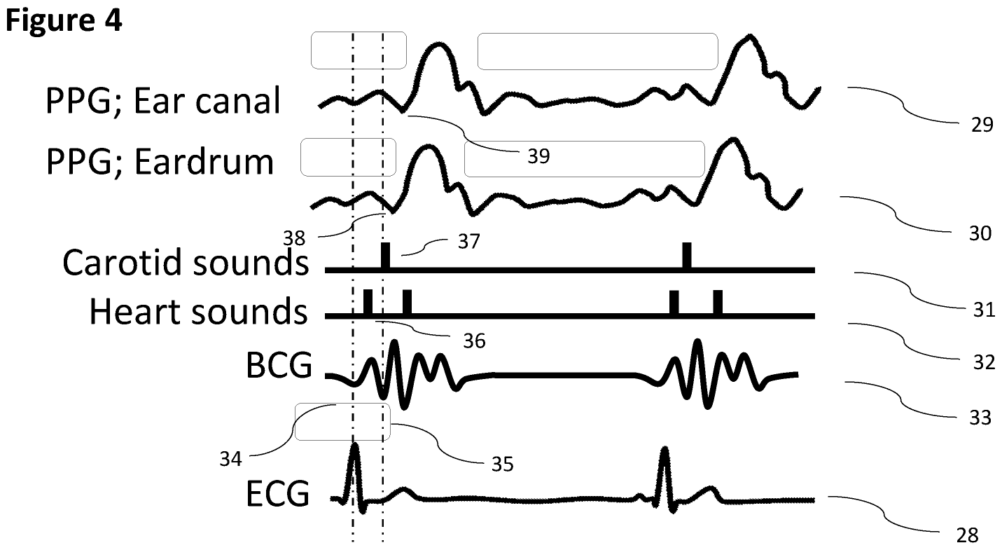

FIG. 4 is an indicative illustration of synchronous physiological signals from embodiments of the invention, with examples of output from some sensors; pulse waveform from PPG directed to ear canal wall 29, pulse waveform from PPG directed to eardrum 30, sound signal from microphone in ear-canal (or held against neck or chest), detecting carotid or other arterial systolic flow 31, or heart sounds 32, from BCG signal 33 detecting ejection impulse from aortic ejection of blood, and from electrodes showing ECG 28 indicating time of onset of electrical activity in the ventricles of the heart 34 (the R wave). Time differences between onset in relationship to the R wave of ECG 34, BCG I-wave 35, first heart sound 36, or carotid blood flow 37, onset of eardrum PPG waveform 38, onset of ear canal PPG wave 39, will be analysed by the processor of the device, and presented on the device, or remotely, to a user as a biological measurement, for example PTT, PAT and difference between internal carotid contributed pulsation and pure external carotid pulse wave. This provides information related to blood pressure, PEP (cardiac contractility) and local blood flow. Data obtained will enable novel analysis and insights into relationship between several cardiac physiological measurements, and other measures and health outcomes that have not previously been investigated, and also provide multiple opportunities to time the cardiac cycle, providing robust measurement of PAT, PTT and other cardiac time intervals.

The invention and embodiments are the only devices and methods providing multiple cardiac physiological measurements, in devices suitable for home, community, hospital and bedside rapid assessment, providing comparison of synchronous measures, allowing determination of relationships between measures that have not previously been measurable in usual clinical care.

In a yet further embodiment the apparatus may incorporate light emitters, which may emit light of single or multiple wavelengths, which may or may not be coherent light, monochromatic light, or laser emitter or emitters. In such an embodiment the sensor of the apparatus may be an optical sensor that detects a range of wavelengths of light, which may be achieved by optical filters, gratings, multiple sensors or sensor arrays, or optoelectronics mechanisms, whereby the sensor(s) response to different wavelengths of light is varied (for example tuneable filters including those incorporating a MEMS Fabry-Perot Interferometer tuneable filter), or camera, which may be of CCD or CMOS sensor type. The sensor detects light reflected and/or scattered from ear structures, including but not limited to the middle ear, ear-drum complex, ear-drum margin and/or ear-canal. For the purpose of this example, the sensor is 3 in the Figures, although it could be any one or more of sensors 2,3,4,5. The output data from such sensors will incorporate data derived from the wavelengths of light received by the sensor or sensors, and from the amplitude of these light wavelengths attributable to absorption by the molecular constituents; a process subsequently referred to as molecular spectroscopy. In this embodiment the spectroscopy data will be analysed by an algorithm of the processor. This will enable comparison with known spectroscopic signals or “signatures” (for example Raman spectrographs) and/or with the signatures of known biological components and other molecules. The processor may output data related to the concentration of glucose in the tissues of the ear-canal wall by analysis of the presence and amplitude of the spectrographic signature of glucose within the reflected light from the ear structure. In a further example the algorithm will provide output data concerning the existence, presence and concentration of one or more other molecules, which may include, but not limited to, oxygen, cholesterol, triglycerides, urea, albumin, lactic acid, CRP (c-reactive protein) and carbon monoxide.

In an alternative, or in addition, the spectroscopy data may be output data related to other biological tissue changes, for example volume of blood perfusion, inflammation and any other change in biological tissue, including blood cells, that affects the characteristics of ear structure biological tissues and secretions, for example ear wax or cerumen. Accumulation of such data over time and/or multiple users will enable future algorithms, for example those derived from machine learning processes, to associate spectrographic data from the sensors with further biological characteristics. In this embodiment the sensor of the apparatus provides non-contact, non-invasive diagnostic data.

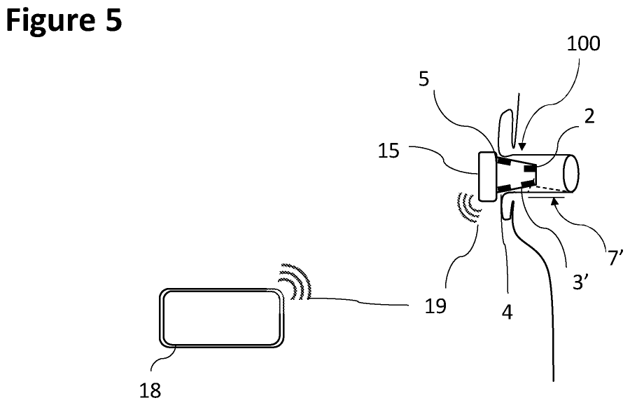

FIG. 5 provides an alternative embodiment which is an ear-based apparatus 100. The ear-based apparatus 100 includes an earpiece component 15′, very similar to earpiece component 15 of FIG. 2, and, thereby, includes several sensors 2,3,4,5. One of the sensors is a Raman spectroscopy emitter/sensor 3′, which is configured to directly illuminate only the medial ⅔^{rds} of the ear-canal 7′ where, according to the invention, preferred reflectance is achieved. The configuration of other sensors 2, 3, 4, 5, may be the same as discussed above for FIGS. 1 and/or 2. Where FIG. 5 differs from FIG. 2 is that there is no flexible component, no hand-held device nor electrode located on the hand-held device; however, the ear-based apparatus 100, excepting those features, does operate very similarly to that embodiment. It communicates with a remote visual graphical display 18, through wireless communications 19 to the processor on the display 18. The ear-based apparatus 100 includes a power source, and associated circuitry for its sensor function, and may include its own processor, memory, etc. in an alternative embodiment. The earpiece component 15′ is very light, inobtrusive and can be worn easily by a user. It is, also, easily secured to the external ear of the user, as it includes an anchor actively contacting at least part of the external ear and/or concha of the user.

In a preferred variant, sensor 2 is an imager, for example a camera or camera and associated light source, for capturing an image of the ear-canal and ear-drum. The image assists in targeting only the medial ⅔^{rds} of the ear-canal 7′ for Raman spectroscopy, and not the ear-drum.

In a further preferred variant, the earpiece component includes adjustment means for controlling and/or locking in situ a position of the earpiece component, at least part of the emitter and/or associated detector in the ear of said user. The adjustment means controls a position of the emitter/sensor 3′, and locks the position to maintain targeting only the medial ⅔^{rds} of the ear-canal 7′.

1-34. (canceled)

35. An apparatus for capturing biometric data from a human or other animal, said apparatus comprising:
an ear portion configured to be received in an ear of said human or other animal,
wherein the ear portion comprises molecular spectroscopy means comprising at least part of an emitter and associated detector that are together configured to capture the biometric data relating to molecular constituents of:
blood passing through the ear of said human or other animal; and/or
ear tissue of said human or other animal.

36. The apparatus as claimed in claim 35, wherein:
i) the spectroscopy means is Raman spectroscopy means, an emitter of which is configured to illuminate a medial ⅔^{rds} of an ear-canal; and/or
ii) at least part of the emitter and associated detector are configured to detect a reflected signal from a/said medial ⅔^{rds} of said ear-canal.

37. The apparatus as claimed in claim 35, wherein the detector is configured to capture biometric data from: an ear drum; an inner (medial) section of an ear canal; an outer (lateral) section of the ear canal; a middle ear; and/or any combinations thereof.

38. The apparatus as claimed in claim 35, wherein the apparatus and/or the ear portion additionally comprises one or more sensor means configured to capture the biometric data relating to one or more of a group comprising sound; motion; temperature; blood pressure; respiratory rate; respiratory rhythm and/or character; pulse; oxygen saturation; appearance of ear structures, eardrum and/or ear contents; heart function; ear-drum response to sound; and/or intentional and/or voluntary movement of middle ear muscles.

39. The apparatus as claimed in claim 35, wherein the apparatus comprises one or more sensor means configured to capture the biometric data from an external ear, pinna, neck, chest, hand, or digit.

40. The apparatus as claimed in claim 35, wherein either:
the apparatus is an earphone, earbud, or ear-mounted apparatus; or
the apparatus comprises a hand-held portion in addition to the ear portion, the hand-held and ear portions being operatively connected for communication therebetween.

41. The apparatus as claimed in claim 35, wherein the apparatus comprises means for securely fastening the ear portion within an ear-canal of a user.

42. The apparatus as claimed in claim 35, wherein the ear portion comprises adjustment means for controlling and/or locking in situ a position of the ear portion, at least part of the emitter and/or associated detector.

43. A method for capturing biometric data from a human or other animal, the method comprising:
locating at least part of an apparatus in an ear of the human or other animal, so as to locate at least part of an emitter and detector of molecular spectroscopy means of the apparatus in the ear;
utilizing the molecular spectroscopy means to capture biometric data relating to molecular constituents of:
blood passing through the ear of the human or other animal; and/or
ear tissue of said human or other animal.

44. The method as claimed in claim 43 comprising:
utilizing Raman spectroscopy to capture biometric data and illuminating a medial ⅔^{rds} of an ear-canal; and/or
utilizing Raman spectroscopy to capture biometric data and detecting a reflected signal from the medial ⅔^{rds} of the ear-canal.

45. The method as claimed in claim 43 comprising:
analyzing the biometric data within the ear portion, an earphone, earbud, or ear-mounted apparatus, or
analyzing the biometric data within a hand-held portion of the apparatus.

46. The method as claimed in claim 43, wherein the emitter of the spectroscopy means emits towards: an ear drum; an inner (medial) section of an ear canal; an outer (lateral) section of the ear canal; a middle ear; and/or any combinations thereof.

47. The method as claimed in claim 43 wherein the biometric data relates to one or more of a group comprising sound; motion; temperature; blood pressure; respiratory rate; respiratory rhythm and/or character; pulse; oxygen saturation; appearance of ear structures, eardrum and/or ear contents; heart function; ear-drum response to sound; and/or intentional and/or voluntary movement of middle ear muscles.

48. The method as claimed in claim 43 comprising detecting the biometric data from an outer ear, pinna, neck, chest, hand, or digit.

49. An apparatus for capturing biometric data from a human or other animal, said apparatus comprising:
an ear portion configured to be received in an ear of said human or other animal,
wherein the ear portion comprises sensor means comprising at least part of an emitter and associated detector of a photoplethysmography sensor means configured to be directed to non-pigmented ear tissue so as to capture the biometric data from said non-pigmented ear tissue.

50. The apparatus as claimed in claim 49, comprising one or more sensor means configured to capture the biometric data relating to sound; motion; temperature; blood pressure; respiratory rate; respiratory rhythm and/or character; pulse; oxygen saturation; appearance of ear structures, eardrum and/or ear contents; heart function; ear-drum response to sound; and/or intentional and/or voluntary movement of middle ear muscles.

51. The apparatus as claimed in claim 49, wherein the sensor means is configured to capture the biometric data from an ear drum; an inner (medial) section of an ear canal; a medial ⅔^{rds} of the ear-canal; an outer (lateral) section of the ear canal; a middle ear; and/or any combinations thereof.

52. The apparatus as claimed in claim 49, wherein the apparatus is configured to further detect the biometric data from an outer ear, pinna, neck, chest, hand, or digit.

53. The apparatus as claimed in claim 49, wherein the apparatus comprises a plurality of sensor means, in which at least two sensor means of the plurality of sensor means are configured to detect the same biometric data from different parts of the ear and/or different parts of the body.

54. The apparatus as claimed in claim 49, wherein the apparatus additionally comprises molecular spectroscopy means, at least part of which is located in the ear portion for capturing the biometric data relating to the molecular constituents of:
blood passing through the ear of said human or other animal; and/or
ear tissue of said human or other animal.

55. The apparatus as claimed in claim 49, wherein the sensor means is configured to be directed to non-pigmented ear tissue of a medial ⅔^{rds} of an ear-canal so as to capture biometric data from said medial ⅔^{rds} of said ear-canal.

56. The apparatus as claimed in claim 49, wherein the photoplethysmography sensor means:
i) comprises a non-contact photoplethysmography sensor means; and/or
ii) is configured to emit and detect multi-wavelength and/or white light.

57. The apparatus as claimed in claim 49 further comprising means for securely fastening the ear portion within an ear-canal of a user.

58. The apparatus as claimed in claim 49, wherein the ear portion comprises adjustment means for controlling and/or locking in situ a position of the ear portion, at least part of the emitter and/or associated detector.

59. A method for capturing biometric data from a human or other animal, the method comprising:
locating at least part of an apparatus in an ear of the human or other animal, so as to locate at least part of an emitter and detector of photoplethysmography sensor means of the apparatus in the ear;
directing the emitter and/or detector to non-pigmented ear tissue; and
detecting and recording the biometric data from said non-pigmented ear tissue.

60. The method as claimed in claim 59 comprising detecting and recording biometric data relating to sound; motion; temperature; blood pressure; respiratory rate; respiratory rhythm and/or character; pulse; oxygen saturation; appearance of ear structures, eardrum and/or ear contents; heart function; ear-drum response to sound; and/or intentional and/or voluntary movement of middle ear muscles.

61. The method as claimed in claim 59 comprising directing the emitter and/or detector to non-pigmented ear tissue of a medial ⅔^{rds} of an ear-canal and detecting and recording biometric data from the medial ⅔^{rds} of the ear-canal.
